(19) United States
(12) Patent Application Publication
(10) Pub. No.: US 2021/0077303 A1
(43) Pub. Date: Mar. 18, 2021

(54) Bandage that elevates over a wound

(71) Applicant: Kevin X Jackson, Suffield, CT (US)

(72) Inventor: Kevin X Jackson, Suffield, CT (US)

(21) Appl. No.: 16/974,111

(22) Filed: Oct. 5, 2020

(51) Int. Cl. A61F 13/02

(52) U.S. Cl. CPC A61F 13/0243; A61F 2013/00272; A61F 13/0206

(57) ABSTRACT

A bandage with a center that elevates over an open wound or desired portion of skin to be covered which forms a dome that has tiny vent holes to allow air inside. The dome allows fresh air to the wound while it is still protected from harmful bacteria, infections, virus' or disease.

### FIELD

The present application relates to a bandage that elevates over a wound and allows fresh air to the wound.

### BACKGROUND

Bandages cover a wound at first to stop the initial bleeding and once the bleeding stops, the bandage is best to be removed to allow fresh air to the wound which helps heal it. This now leaves open flesh exposed to harmful bacteria, virus' and germs. A regular bandage cannot allow air to the wound and also protect it from harmful bacteria, virus' and germs at the same time.

### SUMMARY

By having a bandage that is used after a regular bandage has already stopped the bleeding which can allow air to the wound while it is still healing, it can allow fresh air to help heal it, while at the same time protect it from harmful bacteria, virus', diseases, etc.

In one embodiment the center of the bandage with the blood absorbent cloth which covers the wound, it is elevated just enough to allow air to the wound.

In an embodiment, there are tiny vents to allow air to the wound.

Further objects, features, advantages and properties of the device according to the present application will become apparent from the detailed description.

### BRIEF DESCRIPTION OF THE DRAWINGS

In the following detailed portion of the present description, the teachings of the present application will be explained in more detail with references to the example embodiments, shown in the drawings, in which:

FIG. 1 is an isometric view of an adhesive dressing according to a first embodiment,

FIG. 2 is a reverse upside down view of the bottom of an adhesive dressing according to a second embodiment, and

FIG. 3 is a vertical side view of an adhesive dressing according to a third embodiment.

### DETAILED DESCRIPTION

In the following detailed description, the adhesive dressing according to the teachings for this application in the form of a bandage will be described by the embodiments. It should be noted that although a rectangular shaped bandage is described, the teachings of this application can be used in any size or shape to fit any wound.

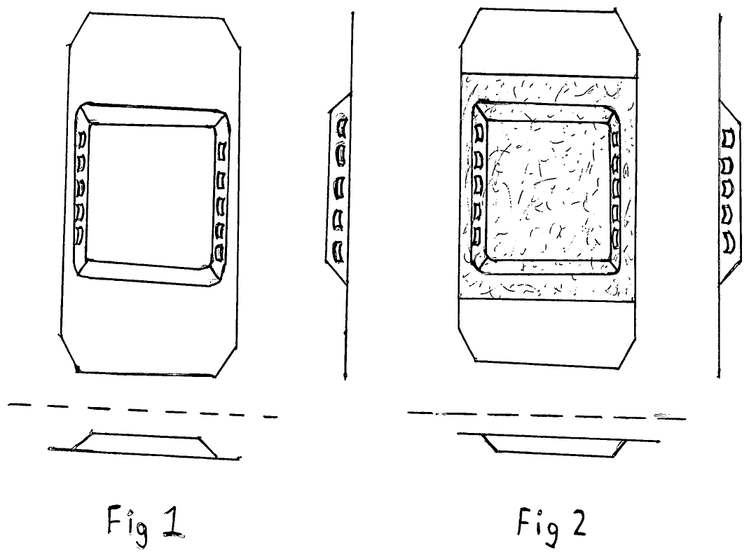

A first embodiment of the adhesive dressing 200, is illustrated in FIG. 1. The bandage 200 comprises a housing 201 with a dome 202, which has tiny vent holes 203 that allows air inside. The bottom of the dome 202 is what would lay over the wound.

In the present embodiment, the entire texture of its front 201 would be made of a thin plastic, latex or any typical bandage material. Only the tiny vent holes 203, would not be covered with the bandage material.

The dome 202 which is elevated by the four sides 202 about a quarter inch, but not limited to any specific size, form a bubble over the wound. The four sides 202 rise up with a slant, but not limited to a slant. The inward slant is only to make it look more presentable. The four sides of the roof 202 are connected with a roof top 205. There are two sides which have vent holes 203 to the left and right that allow air to flow to the inside of the dome 202 which is hollow. Next to the two sides of the dome 202 there are flat surfaces 204 which allows the sides to comfortably lay on the skin.

There are five vent holes 203 on each side of the dome 202 displayed in the illustration but it is not limited to any amount of holes. It also should be noted that the application does not need to have any vent holes.

The present embodiment also has a top flap 201 and a bottom flap 206, which are connected to the dome 202, and the two flaps do not cover the wound, although the reverse sides of them do have about half an inch of blood absorbent cloth in case the blood extends too much beyond the dome 202 which will later be displayed in the next embodiment. However, this application is used after a regular bandage has already stopped the initial bleeding. The top flap 201 and the bottom flap 206 are used to stick the bandage around the wound and the reverse sides of them have a sticky adhesive which stick to the skin around the wound.

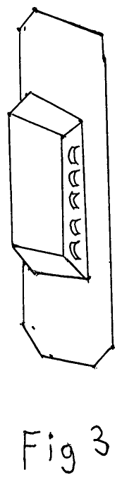

A second embodiment of the elevated adhesive dressing is illustrated in FIG. 2. The elevated adhesive dressing 200 according to the second embodiment is a reverse side view of the bottom of the bandage. The top flap 207 and bottom flap 208 of the reverse side are the sticky adhesive material which sticks the application to the skin. The top flaps inner half 209 and the bottom flaps inner half 211 are blood absorbent cloth. The domes inside 210 is layered with a flexible but firm plastic, no thicker then the plastic of a plastic straw, which is used to keep the structure of the dome in place. The next layer of the domes inside 210 is blood absorbent cloth. The domes inside layers 210 covers the entire inside middle portion except for the vent holes 203. The second half of the bottom flaps inner side 211 would also be blood absorbent cloth.

A third embodiment of the elevated adhesive dressing is illustrated in FIG. 3. The elevated bandage 200 according to a third embodiment is a vertical 3D view of the application. This illustration does not present any additional features but gives a better view and outlook of the presented application.

Although the teachings of the present application are described in detail for the purpose of illustration, it is to be understood that such detail is soley for that purpose, and variations can be made by those skilled in the art without departing from the scope of the teaching.

The teachings of the present application are presented to be used after a regular bandage has already been used but it should be noted that when it is used is at the discretion of the user. It should also be noted that although the application is presented as a rectangular shape, it could also be in any other shape to fit any wound.

Although the application has been described for flesh wounds, it can be used for any other purpose involving the skin, such as pimples, acne, moles, rashes, tattoos, boils, etc.

Features described in the proceeding description may be used in combinations other then the combinations described explicitly herein.

Whilst endeavoring in the foregoing specification to draw attention to these features of the invention believed to be of particular importance, it should be understood that the Applicant claims protection in respect of any patentable features or combination of features hereinbefore referred to and/or shown in the drawings whether or not it be particular emphasis placed thereon.

The term “comprising” as used in the claims does not exclude other elements or steps. The term “a” or “an” used in the claims does not exclude a plurality. A unit or other means may fulfill the functions of several units or means recited in the claims. The term “dome” as used in the claims, means a rising in the structure, elevated from the surface. The term “roof” means the top of the dome.

1. A bandage comprising a center that elevates above the surface.

2. A bandage according to claim 1, wherein the elevated center has four connected sides and a connected roof leaving a hollow inside.

3. A bandage according to claim 2, wherein the elevated dome is layered with latex, thin plastic and blood absorbent cloth.

4. A bandage according to claim 1, wherein the center that elevates above the surface has tiny vent holes.
